(19) United States
(12) Patent Application Publication
(10) Pub. No.: US 2017/0055817 A1
(43) Pub. Date: Mar. 2, 2017

(54) Endoscope Illumination System And Method For Shadow Creation And Improved Depth Perception And Edge Detection

(71) Applicant: Intergrated Medical Systems International, Inc., Birmingham, AL (US)

(72) Inventors: Maciej Dybiec, Cooper City, FL (US); Peter Pal Bodor, Pembroke Pines, FL (US)

(73) Assignee: Intergrated Medical Systems International, Inc., Birmingham, AL (US)

(21) Appl. No.: 15/121,969

(22) PCT Filed: Feb. 19, 2015
(86) PCT No.: PCT/US15/16632

§ 371 Date: Aug. 26, 2016

Related U.S. Application Data
(60) Provisional application No. 61/942,454, filed on Feb. 20, 2014.

(51) Int. Cl. A61B 1/06; A61B 1/00

(52) U.S. Cl. CPC A61B 1/0661; A61B 1/00096

(57) ABSTRACT

A directional illumination system for use with an endoscope for creating shadows in an object field for improving depth perception and contrast. The directional illumination system includes an endoscope distal tip including a plurality of light ports and a plurality of illumination fibers extending within the endoscope distal tip and arranged to direct light through the plurality of light ports onto an object field. At least one light source is operatively coupled to the plurality of illumination fibers for supplying light to the illumination fibers. A switching mechanism is operatively coupled to the plurality of illumination fibers and the at least one light source for selectively delivering light to the illumination fibers. The switching mechanism is configured for activating a first set of illumination fibers that are arranged to direct light through one or more light ports of the plurality of light ports while refraining from activating a second set of illumination fibers, the second set of illumination fibers being arranged to direct light though at least one light port of the plurality of light ports. Activating the first set of illumination fibers without activating the second set of illumination fibers creates shadows in the object field which improves a user's depth perception within the object field.

### FIELD OF INVENTION

The present invention is directed to an endoscope illumination system and method of using same. More particularly, the present invention is directed to a directional illumination system for use with an endoscope for creating shadows in an object field for improving depth perception and contrast.

### BACKGROUND OF INVENTION

Artificial shadow creation methods exist in the field of optics. For example, artificial shadow creation is used in metrology to measure depth and in machine vision applications.

### SUMMARY OF THE INVENTION

The present invention is directed to a directional illumination system for use with an endoscope for creating shadows in an object field for improving depth perception and contrast. According to one aspect of the invention, there is provided an endoscope illumination system including an endoscope distal tip including a plurality of light ports and a plurality of illumination or optical fibers extending longitudinally within the endoscope distal tip and arranged to direct light through the plurality of light ports onto an object field. At least one light source is operatively coupled to the plurality of illumination fibers for supplying light to the illumination fibers. A switching mechanism is operatively coupled to the plurality of illumination fibers and the at least one light source for selectively delivering light to the illumination fibers. The switching mechanism is configured for activating a first set of illumination fibers that are arranged to direct light through one or more light ports of the plurality of light ports while refraining from activating a second set of illumination fibers, the second set of illumination fibers being arranged to direct light though at least one light port of the plurality of light ports.

The first set of illumination fibers can include a first illumination fiber bundle arranged to direct light through a first light port in the distal tip, a second illumination fiber bundle arranged to direct light through a second light port in the distal tip and a third illumination fiber bundle arranged to direct light through a third light port in the distal tip. Additionally, the second set of illumination fibers can include a fourth illumination fiber bundle arranged to direct light through a fourth light port in the distal tip. Preferably, the first illumination fiber bundle, the second illumination fiber bundle, the third illumination fiber bundle and the fourth illumination fiber bundle are each independently activatable. This can be accomplished by providing the switching mechanism with a switch for each of the first, second, third and fourth illumination fiber bundles.

In use, a front end of the endoscope distal tip is faced towards an object to be illuminated and a shadow is created that extends from the object by activating the first set of illumination fibers without activating the second set of illumination fibers. Creation of the shadow improves a user's depth perception within the object field. The endoscope illumination system can also be used to improve edge detection of an area of stained biological tissue by facing a front end of the endoscope distal tip towards the area of stained tissue and activating the first set of illumination fibers without activating the second set of illumination fibers. Thereafter, the second set of illumination fibers is activated without activating the first set of illumination fibers. Depending on the type of dye used to stain the tissue, the at least one light source may emits visual spectrum light or non-visual spectrum light.

According to another aspect of the invention, there is provided an endoscope illumination system including an endoscope distal tip including a first light port and a second light port, a first illumination member within the endoscope distal tip that is arranged to direct light through the first light port onto an object field and a second illumination member within the endoscope distal tip that is arranged to direct light through the second light port onto the object field. At least one light source is operatively coupled to the first illumination member and the second illumination member for providing light that can be transmitted by the illumination members. The light source may include a visual spectrum light source, a non-visual spectrum light source or both the visual spectrum light source and the non-visual spectrum light source A switching mechanism operatively coupled to the first illumination member, the second illumination member and the at least one light source is provided which includes a first switch adapted and arranged to selectively activate and deactivate the first illumination member and a second switch adapted and arranged to selectively activate and deactivate the second illumination member independently of activation of the first illumination member by the first switch.

The endoscope illumination system can include a third light port, a third illumination member within the endoscope distal tip that is arranged to direct light through the third light port onto the object field, a fourth light port and a fourth illumination member within the endoscope distal tip that is arranged to direct light through the fourth light port onto the object field. In this embodiment, the switching mechanism can include third switch and a fourth switch that are adapted and arranged to selectively activate and deactivate the third and fourth illumination members, respectively, independently of activation of the first illumination member by the first switch.

According to yet another aspect of the invention, there is provided a method of using an endoscope including providing an endoscope including an endoscope illumination system having a plurality of selectively activatable illumination members, the plurality of selectively activatable illumination members being arranged within a distal tip of the endoscope to direct light onto an object field, and activating a first selectively activatable illumination member of the plurality of selectively activatable illumination members without activating a second selectively activatable illumination member of the plurality of selectively activatable illumination members. By activating the first selectively activatable illumination member without activating the second selectively activatable illumination member, shadows are created in the object field based upon the morphology of the surface being illuminated. Further, when the surface being illuminated includes stained and unstained areas of biological tissue, activating the first selectively activatable illumination member without activating the second selectively activatable illumination member improves detection of an edge of the area of stained biological tissue in the object field. Edge detection is further improved by activating the second selectively activatable illumination member without activating the first selectively activatable illumination member. Improvement in edge detection comes from direct comparison/overlay of two (first and second selectively activatable illumination) obtained images where the only common feature should be the edge.

### BRIEF DESCRIPTION OF THE FIGURES

FIG. 1 is a perspective view of a front end of an endoscope distal tip in accordance with the present invention displaying multiple light ports for controlled directional illumination.

FIG. 2 is a perspective view of a front end of an endoscope distal tip in accordance with the present invention displaying fiber optic bundles located within the multiple light ports.

FIG. 3 is an elevational view of the front end of the endoscope distal tip of FIG. 1 with schematic drawings illustrating independently activated switch mechanisms and power sources for selectively powering directional illumination bundles that are associated with the lights ports.

FIG. 4A is a perspective view of the endoscope distal tip of FIG. 1 illustrating directional illumination and shadow creation within an object field by selectively powering light bundles to create a leftward extending shadow.

FIG. 4B is a perspective view of the endoscope distal tip of FIG. 1 illustrating directional illumination and shadow creation within an object field by selectively powering light bundles to create a rightward extending shadow.

FIG. 5A is a perspective view of the endoscope distal tip of FIG. 1 illustrating directional illumination and shadow creation within an object field by selectively powering light bundles to create a shadow and expose features that are not visible in direct illumination.

FIG. 5B is a perspective view of the endoscope distal tip of FIG. 1 illustrating directional illumination and shadow creation within an object field by selectively powering light bundles to create a extending shadow.

FIG. 6A is a perspective view of the endoscope distal tip of FIG. 1 illustrating directional illumination within an object field by selectively powering light bundles for enhanced edge detection between a stained area and a non-stained area.

FIG. 6B is a perspective view of the endoscope distal tip of FIG. 1 illustrating directional illumination within an object field by selectively powering light bundles for enhanced edge detection between a stained area and a non-stained area.

### DETAILED DESCRIPTION

The present invention is directed to a directional illumination system for creating shadows in an object field for improving depth perception and contrast. Directional illumination is based on the ability to control a light source in a manner that illuminates an object from a predetermined direction, e.g., from a first lateral side, a second lateral side, a top side or a bottom side, in order to create well-defined shadows. Doing so enhances a user's depth perception within the object field. According to the present invention, directional illumination is accomplished by providing multiple light ports that contain respective optical fiber bundles that are connected with respective light sources that can be switched on/off independently, arranging the light ports so that light from the optical fiber bundles strikes an object in an object field at different angles and activating all but at least one of the optical fiber bundles. Directional illumination can also be accomplished by providing a whole circular light bundle that is divided into sections that can be switched on/off independently.

In addition to improving depth perception and contrast, the directional illumination system of the present invention can be used to improve zone differentiation in fluorescence imaging techniques. Delivering light to fluorescent dyes from different angles using the present invention can improve accurate border analysis and detection of stained areas vs. unstained areas in the object field.

The primary filed of application of the directional illumination system of the present invention is endoscopy. Controlled shadow creation can be used in endoscopy to enhance depth perception of three dimensional objects in the object field. Improved depth perception is desired in endoscopy since objects observed through an endoscope typically provide poor depth perception since they can only be observed from the front of the object with very little space between the distal end of the endoscope and object. The present invention is applicable in the imaging of complex surface morphologies where direct frontal illumination, as is currently provided in endoscopy field, might not reveal all the necessary detail. Further, the present invention can improve contrast where surfaces in the object field are shiny and/or reflective by adding or enhancing shadows which expose contours that might not be visible in uniform frontal illumination.

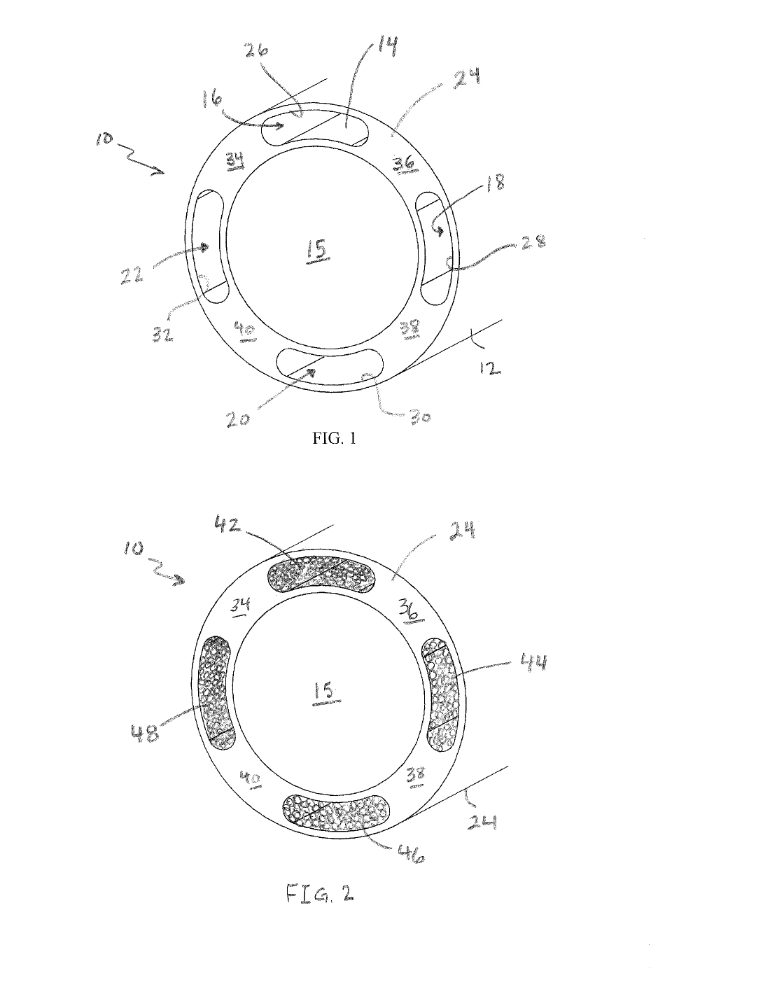

Referring to FIG. 1, there is depicted an endoscope distal tip 10 in accordance with the present invention. Distal tip 10 includes a continuous outer wall 12, a continuous inner wall 14 and a primary channel 14 defined by inner wall 14. Primary channel 15 is typically used to extend surgical instruments through the endoscope and into an object field. Extending between outer wall 12 and inner wall 14 are four illumination channels 16, 18, 20 and 22 that extend longitudinally through distal tip 10 and open through a front face 24 of the distal tip at respective light ports 26, 28, 30 and 32. Illumination channels are sealed from one another by ribs 34, 36, 38 and 40 and arranged equidistantly around front face 24.

Referring to FIG. 2, a plurality of optical fibers extends longitudinally through distal tip 10. The optical fibers are divided into four optical fiber bundles 42, 44, 46 and 48 which extend through respective illumination channels 16, 18, 20 and 22. Optical fiber bundles 42, 44, 46 and 48 are arranged within respective illumination channels 16, 18, 20 and 22 to direct light through respective light ports 26, 28, 30 and 32 and front face 24 of distal tip 10 onto an object field. The placement of light ports 26, 28, 30 and 32 around front face 24 ensures that light emitted by optical fiber bundles 42, 44, 46 and 48 strikes objects with the object field from different angles.

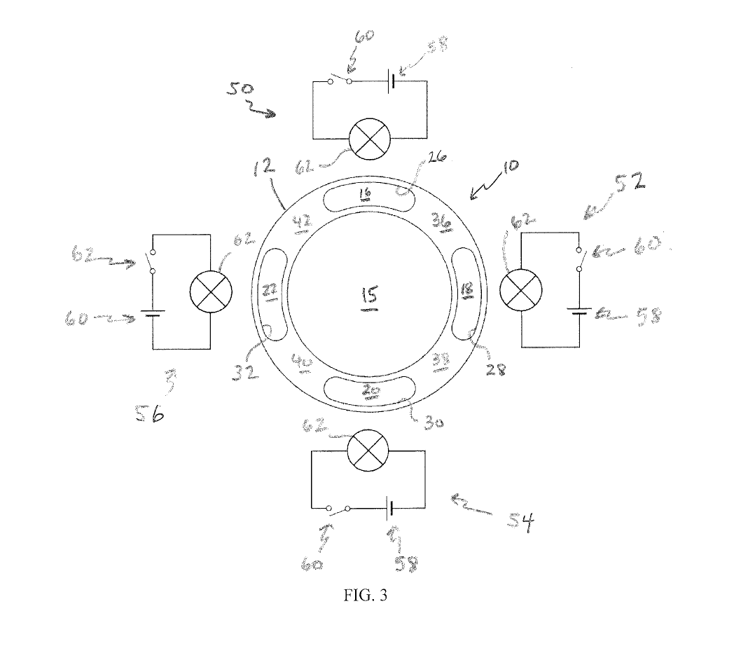

Referring to FIG. 3, optical fiber bundles 42, 44, 46 and 48 receive light from independently controllable light sources 50, 52, 54 and 56 which allows light to be selectively transmitted to and through each of optical fiber bundles 42, 44, 46 and 48. Each of light sources 50, 52, 54 and 56 includes a power source 58, an independently actuatable switch 60 and a light generator 62. Depending on the object field being observed and the surgical procedure being performed, light generator 62 may generate light in the visual spectrum or the non-visual spectrum. In certain instances, it may be beneficial that some of light sources 50, 52, 54 and 56 emit light in the visual spectrum, while other others emit light in the non-visual spectrum. As an alternative to using four independently controllable light sources to provide light independently to each of optical fiber bundles 42, 44, 46 and 48, the present invention may rely upon a single light generator in combination with a light splitter, e.g., one or more prisms, and set of shutters for selectively blocking the transmission of light from the single light generator to the optical fiber bundles.

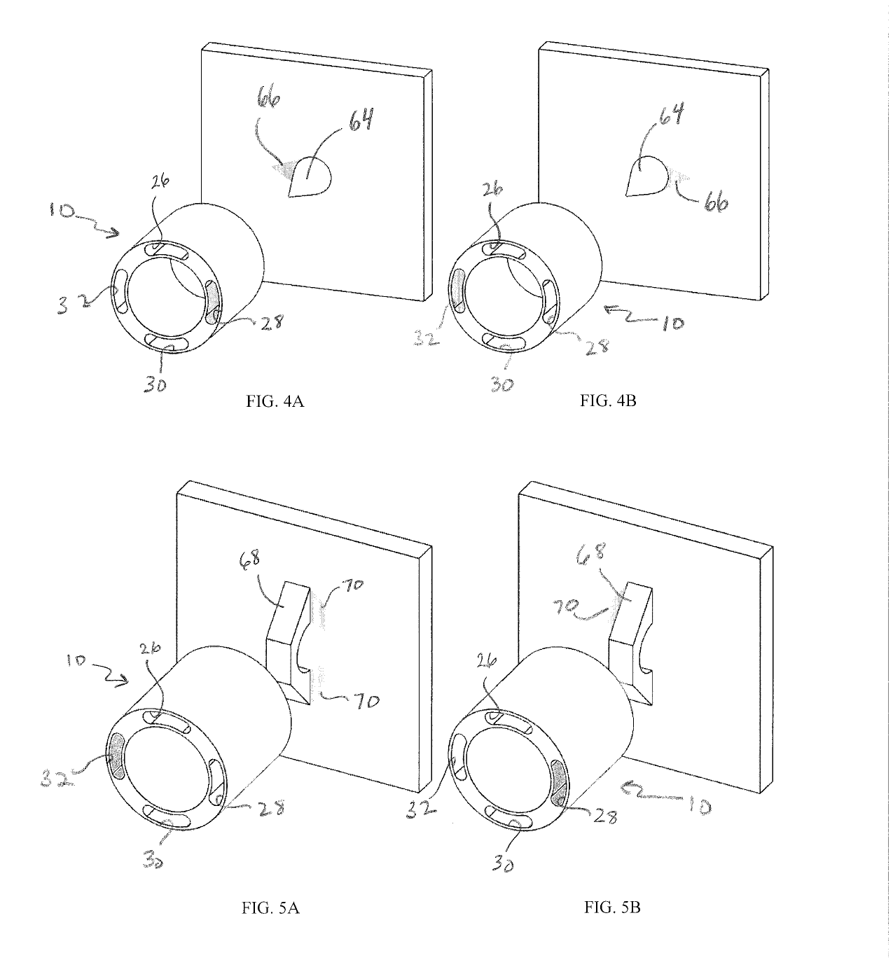

Referring to FIGS. 4A and 4B, there is depicted a method of utilizing a directional illumination system to create shadows in accordance with a first embodiment of the present invention. The method includes providing an endoscope including distal tip 10 with optical fiber bundles 42, 44, 46 and 48 operatively coupled to independently controllable light sources 50, 52, 54 and 56, directing front face 24 of the distal tip toward an object 64 to be illuminated and activating three or less of light generators 62 to thereby emit light from three or less of light ports 26, 28, 30 and 32. As shown in FIG. 4A, light is emitted from a single light port 28, while no light is emitted by light ports, 26, 30 and 32. This accomplished by utilizing switch 60 of light source 52 to activate light generator 62 to transmit light to optical fiber bundle 44, while the switches of light sources 50, 54 and 56 remain in an off position. With light emitting only from light port 28, a shadow 66 is cast that extends leftward from object 64. The existence of shadows 66 adds perspective to the image of object and imparts improved depth perception to the user. Perspective and depth perception are further improved by independently activating other light sources 50, 54 and 56 to form shadows that extend rightward, as depicted in FIG. 4B, upward by activating light source 54 only or downward by activating only light source 50 only. By selectively alternating between the various light sources and thereby the angle by which light strikes object 64, a user can better analyze and visually perceive object 64.

Referring to FIGS. 5A and 5B, there is depicted a method of utilizing a directional illumination system to reveal morphology features that are easily detectable in shadow envelopes in accordance with the present invention. The method includes providing an endoscope including distal tip 10 with optical fiber bundles 42, 44, 46 and 48 operatively coupled to independently controllable light sources 50, 52, 54 and 56, directing front face 24 of the distal tip toward an object 64 to be illuminated and activating three or less of light generators 62 to thereby emit light from three or less of light ports 26, 28, 30 and 32. As shown in FIG. 5A, light is emitted from a single light port 32, while no light is emitted by light port 26, 28 and 30. This accomplished by utilizing switch 60 of light source 56 to activate light generator 62 to transmit light to optical fiber bundle 48, while the switches of light sources 50, 52 and 54 remain in an off position. With light emitting only from light port 32, a shadow 66 is cast that extends rightward from object 64. The existence of shadows 66 adds perspective to the image of object and imparts improved depth perception to the user. Perspective and depth perception are further improved by independently activating other light sources 50, 52 and 54 to form shadows that extend leftward, as depicted in FIG. 5B, upward by activating light source 54 only or downward by activating only light source 50 only. By selectively alternating between the various light sources and thereby the angle by which light strikes object 64, a user can better analyze and visually perceive object 64.

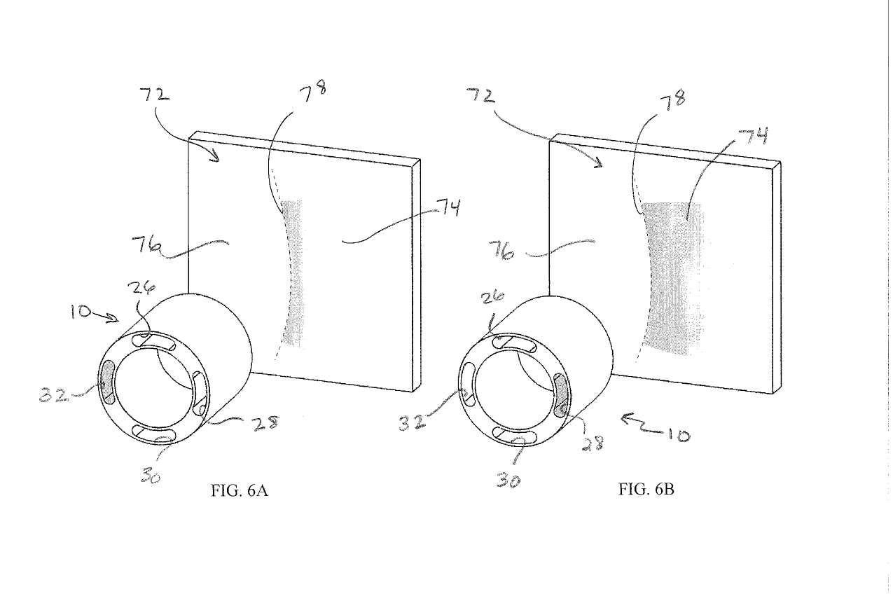

Referring to FIGS. 6A and 6B, there is depicted a method of utilizing a directional illumination system to improve edge detection of a stained biological tissue within an object field in accordance with the present invention. The method includes providing an endoscope including distal tip 10 with optical fiber bundles 42, 44, 46 and 48 operatively coupled to independently controllable light sources 50, 52, 54 and 56, directing front face 24 of the distal tip toward a specimen including a biological tissue that includes a stained portion 74 and an unstained portion 76 and activating three or less of light generators 62 to thereby propagate light from three or less of light ports 26, 28, 30 and 32. As shown in FIG. 6A, light is propagated from a single light port 32, while no light is delivered by light ports, 26, 38 and 30. With light propagating from light port 32, an edge 78 can be observed between the stained portion 74, which emits light due to fluorescence, and the unstained portion 76, which emits little to no light. Additionally, depending on the surface morphology, shadows may be cast adding to the user's depth perception in the object field. Detection and analysis of the of edge 78 is improved by independently activating light source 52, which directs light at edge 78 from different angle than the light emitted from light source 66. With the use of independent light ports 26, 28, 30 and 32, it is possible to create and/or force shadows in one predefined direction that allows for better surface morphology assessment. If a shadow in an expected predefined direction cannot be found, sequential scan with all light ports and consecutive image analysis can be used to determine in what direction surface features are oriented. Analysis of all shadow patterns from sequential illumination provides additional information about feature orientation with respect to the tip of endoscope.

As will be apparent to one skilled in the art, various modifications can be made within the scope of the aforesaid description. Such modifications being within the ability of one skilled in the art form a part of the present invention and are embraced by the claims below. For example, while the embodiments disclosed in this application include four light ports with four associated optical fiber bundles, the invention is not limited to four ports and four optical fiber bundles. It is anticipated that the invention may include more or less than four ports and four optical fiber bundles.

1. An endoscope illumination system comprising:
an endoscope distal tip including a plurality of light ports,
a plurality of illumination fibers extending longitudinally within the endoscope distal tip and arranged to direct light through the plurality of light ports onto an object field,
at least one light source operatively coupled to the plurality of illumination fibers, and
a switching mechanism operatively coupled to the plurality of illumination fibers and the at least one light source,
wherein the switching mechanism is configured for activating a first set of illumination fibers of the plurality of illumination fibers, the first set of illumination fibers being arranged to direct light through one or more light ports of the plurality of light ports while refraining from activating a second set of illumination fibers of the plurality of illumination fibers.

2. The endoscope illumination system according to claim 1 wherein the second set of illumination fibers is arranged to direct light though at least one light port of the plurality of light ports.

3. The endoscope illumination system according to claim 1 wherein illumination fibers are optical fibers.

4. The endoscope illumination system according to claim 1 wherein the one or more light ports include a first light port, a second light port and a third light port and the first set of illumination fibers includes a first illumination fiber bundle arranged to direct light through the first light port, a second illumination fiber bundle arranged to direct light through the second light port and a third illumination fiber bundle arranged to direct light through the third light port.

5. The endoscope illumination system according to claim 4 wherein the one or more light ports include a fourth light port and the second set of illumination fibers includes a fourth illumination fiber bundle arranged to direct light through the fourth light port.

6. The endoscope illumination system according to claim 5 wherein the first illumination fiber bundle, the second illumination fiber bundle, the third illumination fiber bundle and the fourth illumination fiber bundle are each independently triggered.

7. The endoscope illumination system according to claim 1 wherein the switching mechanism includes at least two switches.

8. The endoscope illumination system according to claim 1 wherein the at least one light source includes at least light sources.

9. A method of illuminating an object comprising providing an endoscope including the endoscope illumination system of claim 1, facing a front end of the endoscope distal tip towards the object and creating a shadow extending from the object by activating the first set of illumination fibers without activating the second set of illumination fibers.

10. A method of detecting an edge of an area of stained biological tissue comprising providing an endoscope including the endoscope illumination system of claim 1, facing a front end of the endoscope distal tip towards the area of stained tissue and activating the first set of illumination fibers without activating the second set of illumination fibers.

11. The method according to claim 10 further comprising activating the second set of illumination fibers without activating the first set of illumination fibers.

12. The method according to claim 10 wherein the at least one light source emits visual spectrum light or non-visual spectrum light.

13. An endoscope illumination system comprising:
an endoscope distal tip including a first light port and a second light port,
a first illumination member within the endoscope distal tip that is arranged to direct light through the first light port onto an object field,
a second illumination member within the endoscope distal tip that is arranged to direct light through the second light port onto the object field,
at least one light source operatively coupled to the first illumination member and the second illumination member, and
a switching mechanism operatively coupled to the first illumination member, the second illumination member and the at least one light source,
wherein the switching mechanism includes a first switch adapted and arranged to selectively activate and deactivate the first illumination member and a second switch adapted and arranged to selectively activate and deactivate the second illumination member independently of activation of the first illumination member by the first switch.

14. The endoscope illumination system according to claim 13 further comprising a third light port and a third illumination member within the endoscope distal tip that is arranged to direct light through the third light port onto the object field wherein the third illumination member is operatively coupled with the at least one light source and the switching mechanism, the switching mechanism including a third switch adapted and arranged to selectively activate and deactivate the third illumination member independently of activation of the first illumination member by the first switch.

15. The endoscope illumination system according to claim 14 further comprising a fourth light port and a fourth illumination member within the endoscope distal tip that is arranged to direct light through the fourth light port onto the object field wherein the fourth illumination member is operatively coupled with the at least one light source and the switching mechanism, the switching mechanism including a fourth switch adapted and arranged to selectively activate and deactivate the fourth illumination member independently of activation of the first illumination member by the first switch.

16. The endoscope illumination system according to claim 15 wherein the at least one light source includes a first light source operatively coupled to the first illumination member, a second light source operatively coupled to the second illumination member, a third light source operatively coupled to the third illumination member and a fourth light source operatively coupled to the fourth illumination member.

17. The endoscope illumination system according to claim 16 wherein the switching mechanism is configured for independently activating each of the first light, the second light source, the third light source and the fourth light source.

18. The endoscope illumination system according to claim 13 wherein the at least one light source includes a visual spectrum light source, a non-visual spectrum light source or both the visual spectrum light source and the non-visual spectrum light source.

19. The endoscope illumination system according to claim 13 wherein the first illumination member includes optical fibers.

20. A method of illuminating an object comprising providing an endoscope including the endoscope illumination system of claim 13, facing a front end of the endoscope distal tip towards the object and creating a shadow extending from the object by activating the first illumination member without activating the second illumination member.

21. A method of detecting an edge of an area of stained biological tissue comprising providing an endoscope including the endoscope illumination system of claim 13, facing a front end of the endoscope distal tip towards the area of stained tissue and activating the first illumination member without activating the second illumination member.

22. The method according to claim 21 further comprising activating the second illumination member without activating the first illumination member.

23. A method of using an endoscope comprising:
providing an endoscope including an endoscope illumination system having a plurality of selectively activatable illumination members, the plurality of selectively activatable illumination members being arranged within a distal tip of the endoscope to direct light onto an object field, and
activating a first selectively activatable illumination member of the plurality of selectively activatable illumination members without activating a second selectively activatable illumination member of the plurality of selectively activatable illumination members.

24. The method according to claim 23 wherein activating the first selectively activatable illumination member without activating the second selectively activatable illumination member creates a shadow in the object field.

25. The method according to claim 23 wherein activating the first selectively activatable illumination member without activating the second selectively activatable illumination member improves detection of an edge of an area of stained biological tissue in the object field.

26. The method according to claim 25 wherein activating the second selectively activatable illumination member without activating the first selectively activatable illumination member further improves detection of the edge of the area of stained biological tissue in the object field.

27. The method according to claim 23 further comprising activating a third selectively activatable illumination member of the plurality of selectively activatable illumination members without activating the second selectively activatable illumination member.

28. The method according to claim 27 further comprising activating a fourth selectively activatable illumination member of the plurality of selectively activatable illumination members without activating the second selectively activatable illumination member.

29. The method according to claim 23 further comprising arranging the first selectively activatable illumination member to direct light through a first light port of the distal tip and arranging the second selectively activatable illumination member to direct light through a second light port of the distal tip.
